(19) United States
(12) Patent Application Publication
(10) Pub. No.: US 2025/0375308 A1
(43) Pub. Date: Dec. 11, 2025

(54) KNEE COMPRESSION SLEEVES WITH ATTACHABLE RESISTANCE BANDS

(71) Applicant: Kasper Overaa JORGENSEN, Las Vegas, NV (US)

(72) Inventor: Kasper Overaa JORGENSEN, Las Vegas, NV (US)

(21) Appl. No.: 19/189,165

(22) Filed: Apr. 24, 2025

Related U.S. Application Data
(60) Provisional application No. 63/638,147, filed on Apr. 24, 2024.

(51) Int. Cl. A61F 5/01; A63B 21/00; A63B 21/055

(52) U.S. Cl. CPC A61F 5/0109; A63B 21/00061; A63B 21/0555; A63B 21/4039; A63B 2214/00; A63B 2225/00

(57) ABSTRACT

A pair of knee compression sleeves with attachable resistance bands is provided. In particular, the present disclosure provides an improved exercise system for resistance training and physical therapy involving stretchable knee sleeves configured with fastening mechanisms positioned above and below the knee. The sleeves feature strategically placed hook and loop fasteners on the outer sides of each sleeve, allowing users to securely attach circular resistance bands at both the upper and lower leg regions. This configuration enables safe and effective resistance across a wide range of leg movements without restricting mobility or posing tripping hazards. The system enhances exercise performance, supports joint stability, and prevents displacement of resistance bands during use. The sleeves are designed to provide customizable resistance, joint compression, and warmth, making them suitable for use in walking, running, sprinting, football, fitness training, and rehabilitation exercises.

### RELATED APPLICATION

This application claims priority to U.S. Provisional Application No. 63/638,147, filed Apr. 24, 2024, entitled KNEE COMPRESSION SLEEVES WITH ATTACHABLE RESISTANCE BANDS, by Kasper Overaa Jorgensen, the contents of which are incorporated herein by reference.

### TECHNICAL FIELD

The present disclosure relates to the field of athletic gear, specifically knee compression sleeves designed for enhanced mobility and resistance training during physical exercise. M ore particularly, the invention involves knee compression sleeves equipped with attachable resistance bands, providing versatile support and resistance for a wide range of leg movements.

### BACKGROUND

There have been continuous improvements made to exercise equipment aimed at enhancing user experience, safety, and efficacy during physical activities. The current variety of resistance band exercise equipment poses inherent challenges that impact user safety, mobility, and the overall efficacy of lower extremity workouts. Traditional resistance bands present several limitations related to the positioning of bands and potential risks during various physical activities that have yet to be adequately addressed.

In existing exercise equipment, resistance bands anchored at the ankles may contribute to an increased risk of knee and leg injuries. The fixed positioning places strain on the joints during dynamic movements, potentially leading to discomfort, overuse injuries, and a restricted range of motion.

Traditional resistance bands, when attached at ground level, create tripping hazards during physical activities. The proximity to the ground obstructs natural leg movements, limiting the user's ability to engage in exercises without the constant concern of tripping or stumbling. Traditional resistance bands anchored to the ground or ankles can also pose tripping hazards as they easily catch on nearby items on the ground during exercises.

These traditional exercise bands also come with mobility constraints. The fixed attachment points of resistance bands hinder the natural mobility of the legs during various exercises. This limitation affects the user's ability to perform activities such as walking, running, and sprinting with unrestricted movement, compromising the effectiveness of the workout.

Some resistance bands are not affixed to the leg, rather the tension of the band itself is meant to keep the band in place. However, current resistance band configurations often suffer from the issue of bands shifting or displacing during exercise, leading to instability and potential injury. The lack of a secure attachment mechanism can interfere with the user's ability to maintain proper form and execute movements with confidence.

Beyond safety concerns, current resistance band exercise equipment may contribute to an overall suboptimal user experience. Users may find it challenging to seamlessly incorporate resistance bands into their workout routines, limiting the versatility and effectiveness of lower extremity exercises. In light of these challenges, there exists a need for an innovative solution that addresses the limitations associated with traditional resistance bands and exercise equipment. It would be desirable, therefore, to provide a solution which allows users to engage in various physical activities, with the assurance of optimal resistance placement, and the mitigation of longstanding issues related to safety, mobility, and effective resistance training.

### SUMMARY

According to various embodiments herein, knee compression sleeves with attachable resistance bands are introduced. In particular, the present disclosure provides an improved approach to resistance training through knee compression sleeves with attachable resistance bands designed for optimal support, comfort, and versatility during leg-focused exercises. The present disclosure comprises a pair of compression sleeves, each featuring strategically positioned fasteners (e.g., hook and loop fasteners) on the outer sides. The attachable circular resistance bands can be seamlessly integrated with the compression sleeves using the hook and loop fasteners both above and below the knee, which provide secure attachment points for the resistance bands, allowing users to customize the resistance levels and target specific muscle groups.

The knee sleeves are constructed to offer support, warmth, and mobility, ensuring a wide range of leg movements without the limitations of traditional resistance band configurations. The detachable nature of the resistance bands, held in place by the hook and loop fasteners, eliminates the need for carabiners and allows for quick adjustments during workouts. The present disclosure is tailored for individuals engaged in various physical activities, including walking, running, swimming, sprinting, football, fitness training, physical therapy, and more. The strategic placement of resistance bands above and below the knee enhances resistance in crucial directions, optimizing the effectiveness of exercises.

The present invention offers a solution to the limitations of current resistance band exercise equipment. The design ensures user safety, reduces tripping hazards, and enhances overall mobility, providing a versatile and user-friendly solution for individuals seeking optimal support and resistance during leg-focused exercises. Additional features of the present invention are discussed in the detailed description of the preferred embodiment below, and shown in the drawings hereof, and this summary is not meant to limit the scope of the embodiments herein.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates an example of knee compression sleeves with attachable resistance bands according to the present disclosure from a front view;

FIG. 2 illustrates another example of a knee compression sleeve with attachable resistance bands according to the present disclosure from a side view; and

FIG. 3 illustrates an example of knee compression sleeves with attachable resistance bands in use according to the present disclosure.

### DETAILED DESCRIPTION

The present disclosure introduces knee compression sleeves with attachable resistance bands specially designed to avoid injuries and increase mobility while performing leg exercises and increase user convenience while doing particular resistance training exercises. In some embodiments, the knee compression sleeves with attachable resistance bands comprises a pair of compression sleeves, each featuring strategically positioned fasteners (e.g., hook and loop fasteners) on the outer sides. The attachable resistance bands (e.g., looping/circular) can be seamlessly integrated with the compression sleeves using the hook and loop fasteners both above and below the knee, which provide secure attachment points for the resistance bands, allowing users to customize the resistance levels and target specific muscle groups. The knee sleeves are constructed to offer support, warmth, and mobility, ensuring a wide range of leg movements without the limitations of traditional resistance band configurations. The detachable nature of the resistance bands, held in place by the hook and loop fasteners, eliminates the need for carabiners and allows for quick adjustments during workouts. The strategic placement of resistance bands above and below the knee enhances resistance in crucial directions, optimizing the effectiveness of exercises. The design ensures user safety, reduces tripping hazards, and enhances overall mobility, providing a versatile and user-friendly solution for individuals seeking optimal support and resistance during leg-focused exercises. It is to be understood that the invention is not limited in its application to the details of construction and the arrangement of components described hereinafter and illustrated in the figures. Those skilled in the art will recognize that various modifications can be made without departing from the scope of the invention.

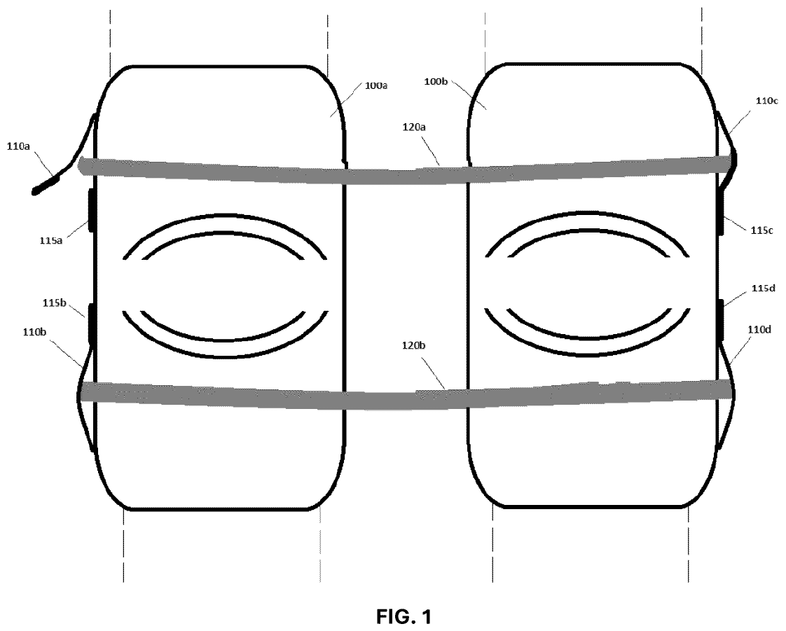

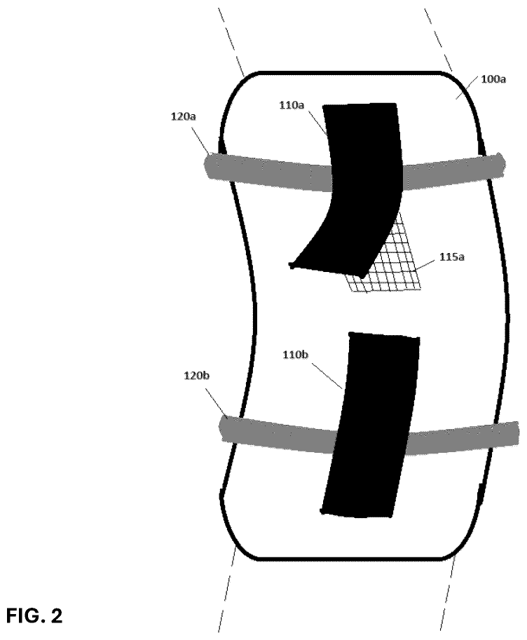

As is generally shown in FIGS. 1-2, the knee compression sleeves 100a-b are comprised of a stretchable yet supportive fabric, such as neoprene, a nylon blend fabric, or any other suitable alternative fabric known in the art. On the outer sides of each knee sleeve are two fastener mechanisms, such as straps 110a-d and correspondingly mating fasteners 115a-d (e.g., hook and loop fasteners), positioned directly above and below the knee. Looping bands (resistance bands 120a-b), made out of material suitable for resistance training such as latex, synthetic rubber, or any other suitable material known in the art, can be positioned around the legs above and/or below the knee and secured in place with the fasteners (e.g., hook and loop fasteners) on the knee sleeves. As shown, strap 110a is illustrated in an open/unfastened position with receiving fastener 115a, while the remainder are shown in closed/fastened position.

The secured resistance bands provide the user with the freedom of movement of their lower body during the performance of exercises without the risk of the resistance bands shifting position. For a general example, when performing side to side squats, as the user bends their legs and steps to the side, the hook and loop fastener keep the resistance bands secured in place so that they do not shift up or down the leg. At the same time, the knee compression sleeve provides support to the knee joint. The top and bottom resistance bands can be different levels of resistance, or the same, as desired.

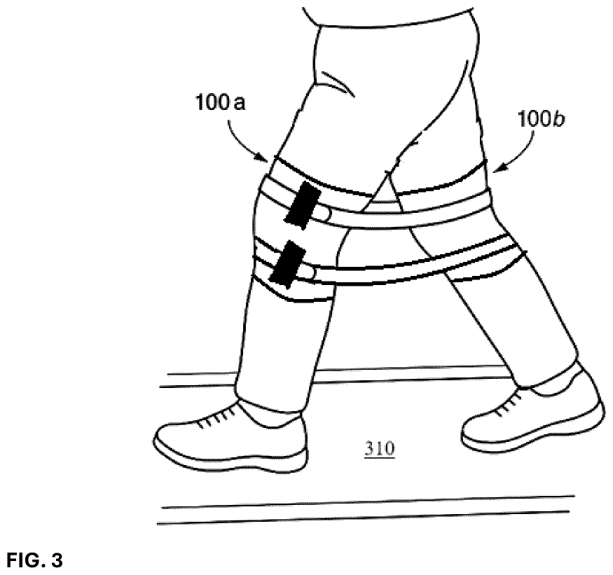

FIG. 3 illustrates an example of the knee compression sleeves 100a-b with attachable resistance bands in use (e.g., on a treadmill 310 or other walking surface) according to the present disclosure, such as while walking on a treadmill for physical therapy.

As will be appreciated, the above examples are intended only for the understanding of certain aspects of the techniques herein and are not limiting in nature. Alternative configurations may be made within the scope of the present disclosure, such as reversing the direction of the fasteners, using buttons and loops for fasteners, using magnets or snaps for fasteners, adding additional support of the looping resistance bands, and so on.

According to one or more specific embodiments herein, an example knee compression sleeve system for resistance training and physical exercise may comprise: a pair of knee compression sleeves, each comprising a stretchable fabric body configured to fit over a knee of a user and surrounding leg region; an upper fastener and a lower fastener on each knee compression sleeve, the upper fastener positioned to be above the knee and the lower fastener positioned to be below the knee; and at least one looping resistance band, wherein the at least one looping resistance band is removably attachable to the upper fastener of each knee compression sleeve or the lower fastener of each knee compression sleeve, thereby securing the at least one looping resistance band around legs of the user in a configuration that provides resistance to movement of the legs during exercise.

In one embodiment, each of the upper fastener and the lower fastener comprises a hook and loop fastening mechanism.

In one embodiment, the stretchable fabric body of each knee compression sleeve comprises neoprene, a nylon blend, or a synthetic elastic material suitable for compression wear.

In one embodiment, the at least one looping resistance band comprises latex, synthetic rubber, or another elastic material configured for resistance training.

In one embodiment, the at least one looping resistance band is configured to be removably attached to the upper fastener of each knee compression sleeve and the lower fastener of each knee compression sleeve simultaneously.

In one embodiment, the upper fastener and the lower fastener of each knee compression sleeve are positioned on lateral sides of the stretchable fabric body.

In one embodiment, each knee compression sleeve further comprises a thermal-retentive inner lining configured to provide warmth and support to a knee joint during exercise.

In one embodiment, the at least one looping resistance band is configured to maintain a secured position during lateral movements, including squats and side lunges, without slipping or rolling along the legs of the user.

In one embodiment, the at least one looping resistance band is adapted for use during physical therapy, sprinting, walking, or sports training.

In one embodiment, at least one of the upper fastener and the lower fastener comprises a magnetic fastener, a snap fastener, or a button-and-loop assembly.

Advantageously, the knee compression sleeves with attachable resistance bands offer a revolutionary solution to the constraints posed by traditional resistance band exercise equipment. Uniquely designed with hook and loop secured circular resistance bands, the knee sleeves provide strategic attachment points both above and below the knee, allowing users to personalize resistance levels and target specific muscle groups during a wide array of leg-focused activities. The innovative design minimizes the risk of knee and leg injuries, reduces tripping hazards, and ensures increased mobility due to the secure hook and loop attachment mechanism. Unlike conventional resistance band configurations, the knee compression sleeves empower users engaged in activities such as walking, running, swimming, sprinting, football, fitness training, and physical therapy, offering unparalleled versatility and comfort. The invention represents a significant advancement, enabling individuals to engage in resistance training with enhanced safety, freedom of movement, and adaptability, ultimately transforming the leg exercise experience. The improved design eliminates the need for ground-based or ankle-anchored resistance bands, streamlining the resistance training process and optimizing safety and comfort for the user.

The foregoing description has been directed to specific embodiments. It will be apparent, however, that other variations and modifications may be made to the described embodiments, with the attainment of some or all of their advantages. Accordingly, this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A knee compression sleeve system for resistance training and physical exercise, the knee compression sleeve system comprising:
a pair of knee compression sleeves, each comprising a stretchable fabric body configured to fit over a knee of a user and surrounding leg region;
an upper fastener and a lower fastener on each knee compression sleeve, the upper fastener positioned to be above the knee and the lower fastener positioned to be below the knee; and
at least one looping resistance band, wherein the at least one looping resistance band is removably attachable to the upper fastener of each knee compression sleeve or the lower fastener of each knee compression sleeve, thereby securing the at least one looping resistance band around legs of the user in a configuration that provides resistance to movement of the legs during exercise.

2. The knee compression sleeve system of claim 1, wherein each of the upper fastener and the lower fastener comprises a hook and loop fastening mechanism.

3. The knee compression sleeve system of claim 1, wherein the stretchable fabric body of each knee compression sleeve comprises neoprene, a nylon blend, or a synthetic elastic material suitable for compression wear.

4. The knee compression sleeve system of claim 1, wherein the at least one looping resistance band comprises latex, synthetic rubber, or another elastic material configured for resistance training.

5. The knee compression sleeve system of claim 1, wherein the at least one looping resistance band is configured to be removably attached to the upper fastener of each knee compression sleeve and the lower fastener of each knee compression sleeve simultaneously.

6. The knee compression sleeve system of claim 1, wherein the upper fastener and the lower fastener of each knee compression sleeve are positioned on lateral sides of the stretchable fabric body.

7. The knee compression sleeve system of claim 1, wherein each knee compression sleeve further comprises a thermal-retentive inner lining configured to provide warmth and support to a knee joint during exercise.

8. The knee compression sleeve system of claim 1, wherein the at least one looping resistance band is configured to maintain a secured position during lateral movements, without slipping or rolling along the legs of the user.

9. The knee compression sleeve system of claim 1, wherein the at least one looping resistance band is adapted for use during physical therapy, sprinting, walking, or sports training.

10. The knee compression sleeve system of claim 1, wherein at least one of the upper fastener and the lower fastener comprises a magnetic fastener, a snap fastener, or a button-and-loop assembly.
